(19) United States
(12) Patent Application Publication
(10) Pub. No.: US 2012/0247481 A1
(43) Pub. Date: Oct. 4, 2012

(54) CERVICAL OCCLUDER

(75) Inventor: Khaled Sakhel, Virginia Beach, VA (US)

(73) Assignee: EASTERN VIRGINIA MEDICAL SCHOOL, Norfolk, VA (US)

(21) Appl. No.: 13/499,765

(22) PCT Filed: Oct. 4, 2010
(86) PCT No.: PCT/US10/51308

§ 371 Date: Jun. 21, 2012

Related U.S. Application Data
(63) Continuation-in-part of application No. 12/572,705, filed on Oct. 2, 2009.

(51) Int. Cl. A61F 6/06

(52) U.S. Cl. 128/830

(57) ABSTRACT

A device for occluding a cervix has an elongated conduit sized and shaped to extend from a proximal end outside the vagina to a distal end near the cervix. A loop sized to fit around an exocervix is provided at the distal end. The loop is sufficiently flexible to be tightened and loosened around the exocervix. The device can include a rod inside the conduit. The rod has a distal end and a proximal end, and the loop is joined to the distal end of the rod. Moving the rod longitudinally relative to the conduit tightens or loosens the loop. The ends of the loop can also extend out through the proximal end of the conduit. Pulling on the ends through the conduit tightens the loop.

This application is a continuation-in-part of U.S. application Ser. No. 12/572,705, filed Oct. 2, 2009, the contents of which are incorporated herein by reference.

### FIELD

This disclosure relates generally to a device for occluding a cervix.

### BACKGROUND

During certain gynecological procedures, the uterine cavity might need to be distended, such as by injecting a gas or a liquid into the uterus. These procedures include hysteroscopic procedures and saline infusion sonography such as the diagnosis and treatment of uterine conditions including, but not limited to, endometrial polyps, abnormal uterine bleeding, uterine fibroids, uterine malformations, sterilization, and infertility work-up. During such distention, if the cervix is not occluded or some other action is not taken, the gas or liquid could escape through the cervix and allow the uterus to contract back to its normal size.

Currently, during procedures where the cervix must be occluded, practitioners can use tenaculums or similar devices to occlude the cervix. However, tenaculums grip and pierce the tissue of the cervix, and this can cause pain and discomfort to the patient.

### SUMMARY

This disclosure describes a device for occluding a cervix inside a vagina. In one aspect, the device has an elongated conduit sized and shaped to extend from a proximal end outside the vagina to a distal end near the cervix. The device also comprises a loop sized to fit around an exocervix. The loop is sufficiently flexible to be tightened and loosened around the exocervix. A portion of the loop is disposed inside the conduit, and a portion of the loop protrudes from the distal end of the conduit.

In some embodiments, the device includes a rod inside the conduit and movable relative to the conduit. The rod has a distal end and a proximal end, and the loop is joined to the distal end of the rod. Moving the rod longitudinally relative to the conduit tightens or loosens the loop. In other embodiments, the ends of the loop extend through the proximal end of the conduit. Pulling on these ends in a proximal direction tightens the loop.

This disclosure also includes a method for occluding a cervix. In this method, a device is inserted into a patient's vagina. The device has an elongated conduit sized and shaped to extend from a proximal end outside the vagina to a distal end near the cervix. The device also comprises a loop sized to fit around an exocervix. The loop is sufficiently flexible to be tightened and loosened around the exocervix. A portion of the loop is disposed inside the conduit and a portion of the loop protrudes from the distal end of the conduit, allowing the loop to be tightened to a closed position and loosened to an open position. The device can additionally comprise a rod disposed inside the conduit and coupled to the loop to allow the practitioner to tighten or loosen the loop around the patient's exocervix. Alternatively, the ends of the loop can extend out through the proximal end of the conduit. These ends may be pulled in a proximal direction to tighten the loop around the patient's exocervix.

The cervical occluding device permits a practitioner to occlude the cervix during gynecological procedures. After the cervix is occluded, the uterus can be distended with a fluid. Then, procedures can be implemented, such as treating endometrial polyps, abnormal uterine bleeding, uterine fibroids, uterine malformations, sterilization, and infertility work-up. The device also can reduce the amount of injury and discomfort a patient experiences and allows for a more flexible environment for a clinician to work in.

### BRIEF DESCRIPTION OF THE FIGURES

FIG. 1 is a side view of an embodiment of a cervical occluding device.

FIG. 2 is a view of the cervical occluding device of FIG. 1 in an operator's hand.

FIG. 3 is a cross-sectional view of the device through lines 3-3 of FIG. 1.

FIG. 4 is a cross-sectional view of the device of FIG. 1-3 through lines 4-4 of FIG. 3.

FIG. 5 shows a speculum inserted into a vagina with a hysteroscope fed through the loop of the cervical occluding device and through the speculum into the uterus.

FIG. 6 shows a speculum inserted into a vagina with a hysteroscope fed through the speculum into the uterus and the cervical occluding device within the speculum with the loop positioned around the exocervix.

FIG. 7 is a side view of another embodiment of a cervical occluding device.

FIG. 8 is a view of the cervical occluding device of FIG. 7 in an operator's hand.

FIG. 9 is a cross-sectional view of the device through lines 9-9 of FIG. 7.

FIG. 10 is a cross-sectional view of the device of FIG. 7-9 through lines 10-10 of FIG. 9.

### DETAILED DESCRIPTION

This disclosure describes a device for occluding a cervix. As used herein, the term “cervix” includes the exocervix. As used herein, the term “about” means ±10%.

As shown in the embodiment illustrated in FIG. 1, the device has an elongated conduit 1 sized and shaped to extend from proximal end 2 outside the vagina to distal end 3 near the cervix when inserted. Conduit 1 is held with a handle 7, which can be designed for left and right handed use, near proximal end 2. Generally, the conduit can range in length from about 4 inches (10 cm) to about 12 inches (30 cm). The conduit can have a uniform cross-sectional diameter, or as shown, can have multiple sections including a larger diameter section 1a and a smaller diameter section 1b. The conduit can include plastic or metal.

A loop 4 extends from distal end 3 of the conduit and is sized to fit around a patient's exocervix. Loop 4 is sufficiently flexible to be tightened and loosened around the exocervix. A portion of loop 4 is disposed inside conduit 1 and a portion of loop 4 protrudes from distal end of the conduit 3. The loop can include any suitable material that is flexible to fit around an exocervix and strong enough to tighten around the cervix, such as a synthetic material such as polypropylene, and can be formed as a mesh.

Referring to FIGS. 1 and 3, a rod 5 is provided inside conduit 1 and extends along much of the length of conduit 1. Rod 5 has a distal end and a proximal end, with loop 4 coupled to rod 5 at the distal end. Moving rod 5 longitudinally relative to conduit 1 tightens or loosens the loop. FIG. 1 shows the loop fully closed in solid lines and fully opened in dashed lines. The rod can include plastic or metal.

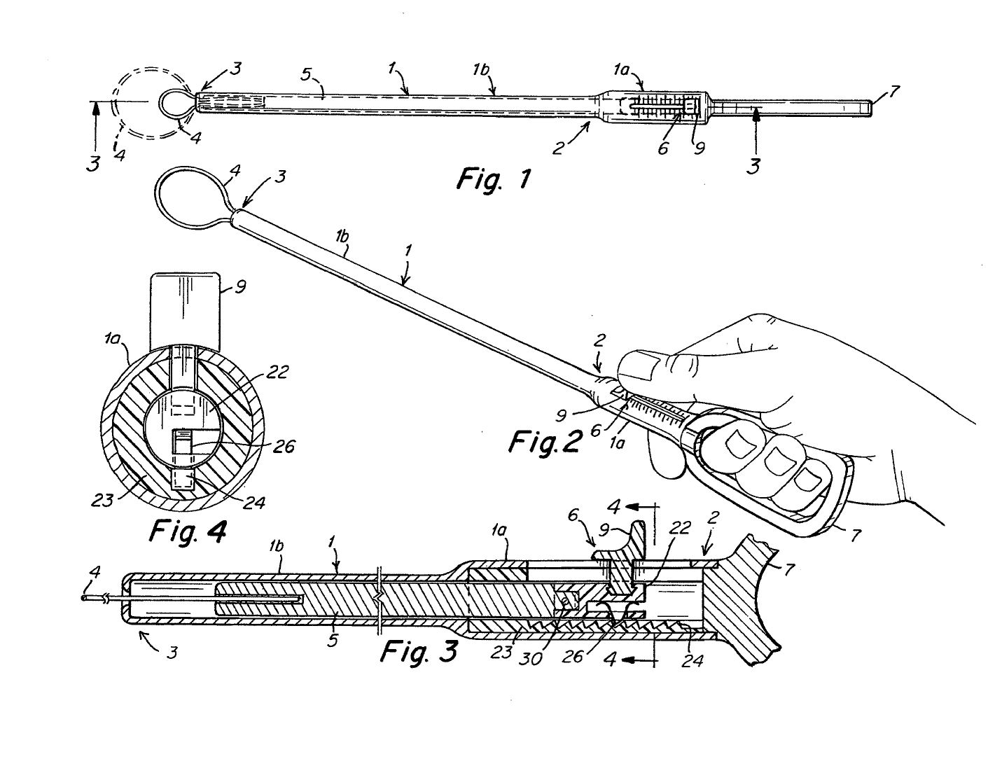

Rod 5 can be coupled to any suitable actuator for moving the rod, desirably an actuator that is manually movable without additional tools. Referring to FIGS. 1-4, one embodiment of such an actuator includes thumbslide 6 that has a tab 9 that protrudes through a slot opening in conduit 1 and allows the thumbslide and rod to be moved longitudinally.

Referring particularly to FIGS. 3 and 4, tab 9 for the thumbslide 6 extends through conduit 1 and into a molded piece 22 coupled to rod 5. Referring to FIG. 3, a pin 30 can be used as a connection between rod 5 and molded piece 22.

The device can include a lock for maintaining a position of the rod relative to the conduit to avoid one from moving longitudinally relative to the other after the loop has been positioned. Referring to FIG. 3, a series of teeth 24 can be disposed along the inside of an insert 23 to conduit 1. Molded piece 22 is coupled to a leaf spring 26 that catches on teeth 24 and holds the position of rod 5 relative to conduit 1 to avoid one from moving longitudinally relative to the other. Other suitable locks for maintaining a position of the rod relative to the conduit to avoid one from moving longitudinally relative to the other can also be used. For example, a threaded rod may extend through an opening in a knob coupled to the proximal end of the rod. A nut can be disposed on the threaded rod, where the nut can be tightened to prevent the rod from moving in a proximal direction. As another example, the slot for the slide can have a series of additional perpendicular slots so that the tab 9 is rotated slightly to fit into a slot to prevent the rod from moving longitudinally.

The resulting occlusion of the cervix by moving rod longitudinally in a proximal direction is reversible. By moving the rod in a distal direction, the loop may be loosened. After the loop is loosened, the loop may be removed from the cervix.

This disclosure also provides a method of occluding a cervix. In this method, a cervical occluding device as described herein is inserted into a patient's vagina. The loop is positioned around the patient's exocervix, and the portion of the loop disposed inside the conduit is moved in a proximal direction, thereby tightening the loop around the exocervix. This tightening can be done before the uterus is distended. This method can further include inserting a hysteroscope or other instrument through the cervix before tightening the loop around the cervix.

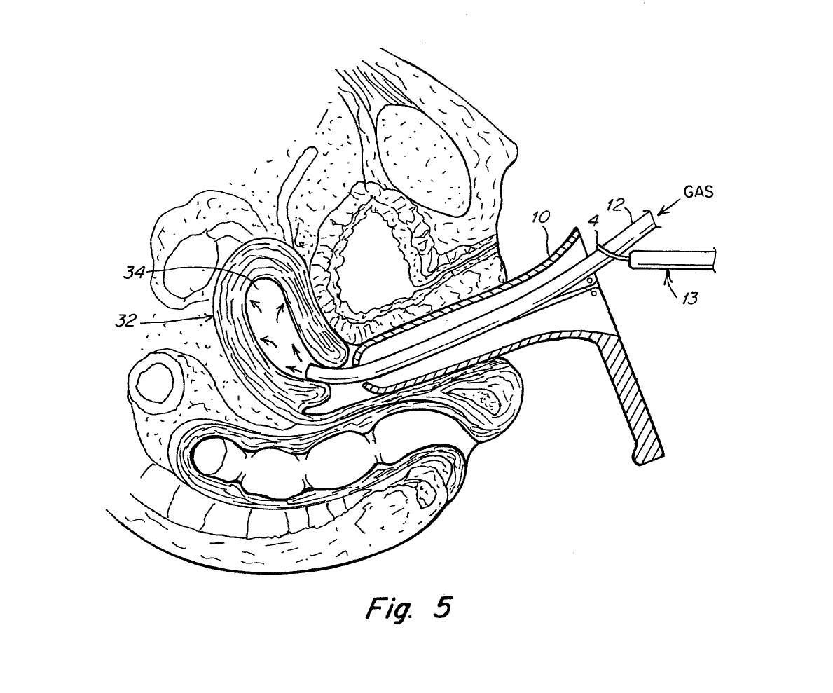

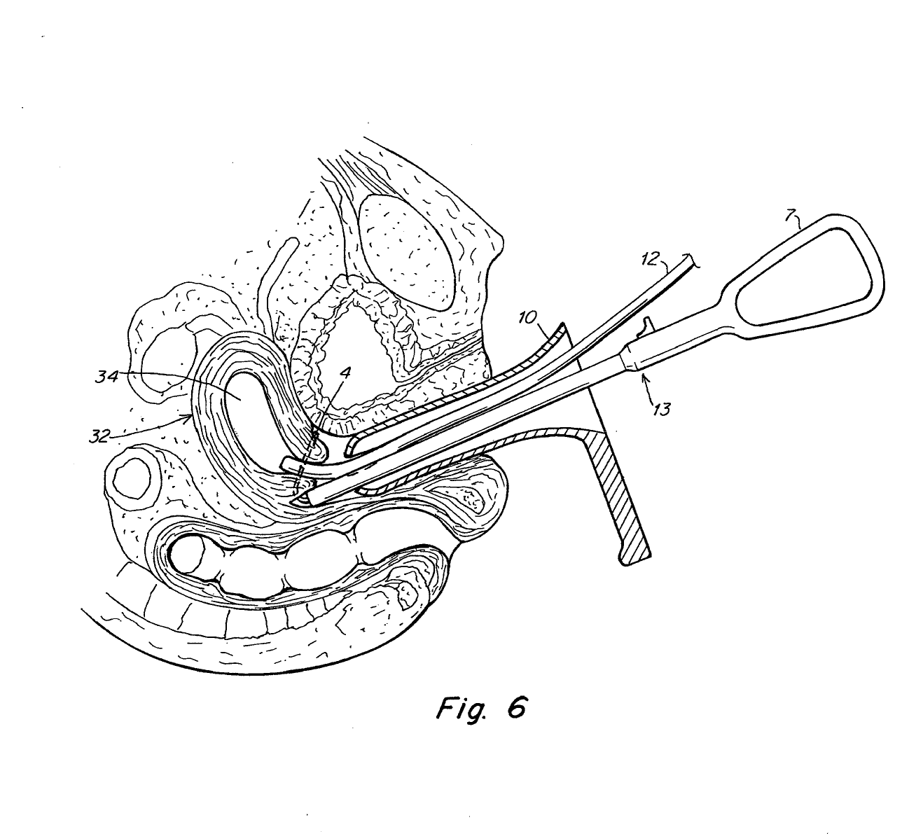

Referring to FIGS. 5 and 6, the cervical occluder, the end of which is shown at 13, is useful during hysteroscopic procedures during which the uterine cavity is distended. During such procedures, a hysteroscope 12 can be introduced through the loop, a speculum 10, the cervix 32, and into uterine cavity 34. Once hysteroscope 12 is introduced to the uterine cavity, then the rod (FIGS. 1-4) can be moved longitudinally in a proximal direction to tighten loop 4 around cervix 32 and hysteroscope 12. The cervical occluder allows the uterine cavity to be distended and maintained without much discomfort to the patient and without injuring the cervix. Once the uterine cavity is distended, the clinician is able to proceed with the hysteroscopic procedure.

As shown in FIG. 6, cervical occluder 13 is fed through speculum 10 and into position proximate to cervix 32 so loop 4 (shown in dashed lines) is positioned around the exocervix. Once loop 4 is in position, cervix 32 can be occluded around hysteroscope 12 by operating the actuator in the occluder, such as by drawing back the thumbslide.

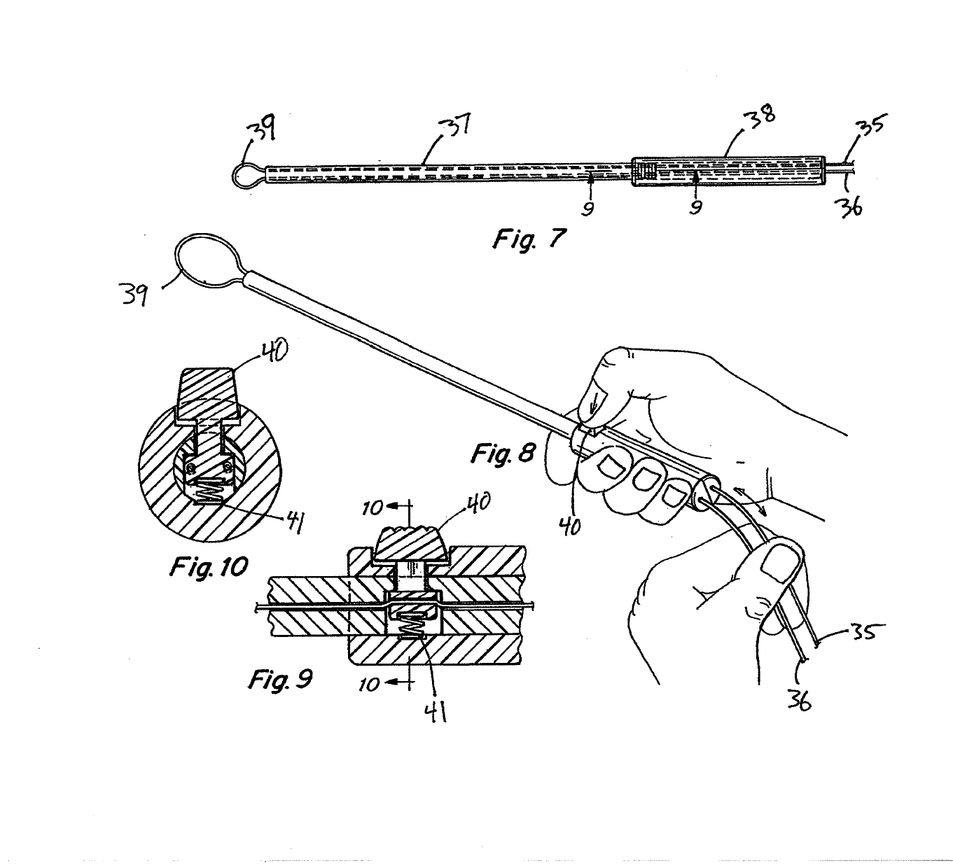

Loop 4 is removed by advancing the rod outwardly relative to the conduit (FIGS. 1-4). The loop should only require a small release of tension to allow it to be removed. The loop can be made from a material that has sufficient flexibility to close around the circumference of the exocervix, while also being sufficiently stiff to allow it to be released. Although FIGS. 5 and 6 show the cervical occluder of FIGS. 1-4, other cervical occluders, including the cervical occluder shown in FIGS. 7-10, can be used in the disclosed methods for occluding a cervix.

Referring to FIGS. 7-10, ends of the loop 35 and 36 can extend out through the proximal end of the device. Ends of the loop 35 and 36 can be disposed in separate openings in conduit 37. Conduit 37 is held by handle 38. Handle 38 can have the same as or, as shown, a larger cross-sectional diameter than conduit 37. To tighten loop 39 around the exocervix, the loop is positioned around an exocervix and ends of the loop 35 and 36 are pulled in a proximal direction through conduit 37 until a desired tightness of loop 39 is reached.

The device can also include a lock near the proximal end of conduit 37 for maintaining the tightness of loop 39. When the lock is unlocked, ends of the loop 35 and 36 can be pulled through conduit 37 to tighten loop 39. Once the desired level of tightness is reached, the lock can be locked to maintain the tightness of loop 39. Referring to FIGS. 8-10, the lock can include a mechanism 40 supported by a spring 41. When mechanism 40 is depressed, ends of the loop 35 and 36 are able to move freely. When mechanism 40 is not depressed, the lock maintains the position of ends of loop 35 and 36 relative to the conduit. Other suitable locks for maintaining a position of ends of the loop relative to the conduit can also be used. For example, the conduit may include a slot perpendicular to the direction of travel of the ends of the loop. An insert can be slid into the slot to pin the ends of the loop in place.

The resulting occlusion of the cervix by depressing the spring-supported mechanism, pulling the ends of the loop in a proximal direction, and then releasing the spring-supported mechanism is reversible. By depressing the spring-supported mechanism and pulling back on the device so that it slides along the portion of the loop disposed inside the conduit, the loop may be loosened. After the loop is loosened, it may be removed from the cervix.

The cervical occluder can be provided as a multi-use product or a single-use disposable product. If multi-use, the design should allow for convenient cleaning and sterilization.

Other embodiments are within the scope of the following claims. For example, while certain materials have been described, others could be used. A particular lock design has been shown, for example, in the embodiment of FIGS. 7-10, but other types of locks could be used.

1. A device for occluding a cervix of a human through a vagina, comprising:
an elongated conduit sized and shaped to extend from a proximal end outside the vagina to a distal end near the cervix;
a loop sized to fit around an exocervix, the loop being sufficiently flexible to be tightened and loosened around the exocervix, wherein a portion of the loop is disposed inside the conduit and a portion of the loop protrudes from the distal end of the conduit; and
wherein moving the portion of the loop disposed inside the conduit towards the proximal end of the conduit tightens the loop.

2. The device of claim 1, further comprising a lock for maintaining a position of the portion of the loop disposed inside the conduit.

3. The device of claim 2, wherein the lock includes a spring.

4. The device of claim 1, wherein the loop includes synthetic mesh.

5. The device of claim 1, wherein the loop includes polypropylene.

6. The device of claim 1, wherein the device is a single use disposable device.

7. The device of claim 1, wherein the conduit further comprises a handle proximate to the proximal end.

8. The device of claim 1, further comprising an actuator for moving the portion of the loop disposed inside the conduit and causing the loop to be tightened or loosened around the exocervix.

9. The device of claim 8, further comprising a lock for maintaining a position of the actuator to inhibit the loop from tightening or loosening after the loop has been positioned.

10. The device of claim 9, wherein the lock includes teeth and a spring.

11. The device of claim 8, wherein the actuator includes a rod disposed inside the conduit, the rod having a distal end and a proximal end, the loop joined to the distal end, and wherein moving the rod longitudinally relative to the conduit tightens or loosens the loop.

12. The device of claim 8, wherein the actuator further comprises a thumbslide coupled to the proximal end of the rod for moving the rod in a longitudinal direction.

13. The device of claim 8, wherein the loop includes synthetic mesh.

14. The device of claim 8, wherein the loop includes polypropylene.

15. The device of claim 8, wherein the device is a single use disposable device.

16. The device of claim 8, wherein the conduit further comprises a handle proximate to the proximal end.

17. A method comprising:
inserting the occluding device of claim 1 into a patient's vagina;
positioning the loop of the occluding device around the patient's exocervix; and
moving the portion of the loop disposed inside the conduit towards the proximal end of the conduit to tighten the loop around the exocervix.

18. The method of claim 17, further comprising distending the patient's uterus cavity after the loop is tightened around the exocervix.

19. The method of claim 17, further comprising inserting into the uterus a hysteroscope prior to tightening the loop around the exocervix, and then tightening the loop around the exocervix such that the hysteroscope remains extending into the uterus.

20. The method of claim 17, further comprising, prior to the inserting, inserting a speculum into the patient's vagina, and then inserting the occluding device through the speculum.

21. The method of claim 18, wherein the occluding device of claim 1 further comprises an actuator for moving the portion of the loop disposed inside the conduit.
